(19) United States
(12) Patent Application Publication
(10) Pub. No.: US 2025/0111947 A1
(43) Pub. Date: Apr. 3, 2025

(54) METHOD, INFORMATION PROCESSING APPARATUS, SYSTEM, AND PROGRAM

(71) Applicant: Sompo Care Inc., Shinagawa-ku, Tokyo (JP)

(72) Inventors: Yohei MIYAGATA, Shinjuku-ku, Tokyo (JP); Keiko HORI, Shinjuku-ku, Tokyo (JP); Masaki TORII, Shinagawa-ku, Tokyo (JP)

(73) Assignee: Sompo Care Inc., Shinagawa-ku, Tokyo (JP)

(21) Appl. No.: 18/838,716

(22) PCT Filed: Feb. 22, 2023
(86) PCT No.: PCT/JP2023/006596

§ 371 Date: Aug. 15, 2024

(30) Foreign Application Priority Data
Mar. 3, 2022 (JP) 2022-032935

(51) Int. Cl. G16H 50/30; A61B 5/00; G16H 10/60; G16H 20/10

(52) U.S. Cl. CPC G16H 50/30; A61B 5/4848; G16H 10/60; G16H 20/10

(57) ABSTRACT

Technology related to maintaining the physical condition of residents in residential facilities is improved. A controller 11 of an information processing apparatus 10 acquires an ingestion history of ingested substances and a change in health level of a resident in a facility, quantitatively analyzes an effect on health level for each ingested substance or combination of ingested substances based on an ingestion history and change in health level of a plurality of residents, and outputs information on the effect on health level for each ingested substance or combination of ingested substances. The change in health level includes at least one of a change in amount of sleep, a change in food intake, a change in accident rate, a change in respiratory rate, and a change in heart rate.

### TECHNICAL FIELD

The present disclosure relates to a method, an information processing apparatus, a system, and a program.

### BACKGROUND

Technology related to management of prescription drugs has been developed. For example, Patent Literature (PTL) 1 discloses technology for a prescription drug management system that prescribes the appropriate type and dose of drugs in a nursing care facility based on living level evaluation data, obtained by periodically evaluating the level of living of patients and users in accordance with behavior patterns in their daily life, and prescription drug data indicating the drugs prescribed to the residents. The proposal of changes to the prescription drug data with reference to drug risk data such as adverse effect data is also disclosed.

### CITATION LIST

### Patent Literature

PTL 1: JP 2021-64294 A

### SUMMARY

### Technical Problem

There is room for improvement in technology for managing residents' prescription drugs and diet and maintaining residents' physical condition in nursing homes or other residential facilities for the elderly.

In light of these circumstances, it is an aim of the present disclosure to improve technology related to maintaining the physical condition of residents in residential facilities.

### Solution to Problem

A method according to an embodiment of the present disclosure is a method to be executed by an information processing apparatus, the method including acquiring an ingestion history of ingested substances and a change in health level of a resident in a facility; quantitatively analyzing an effect on health level for each ingested substance or combination of ingested substances based on an ingestion history and change in health level of a plurality of residents; and outputting information on the effect on health level for each ingested substance or combination of ingested substances, wherein the change in health level includes at least one of a change in amount of sleep, a change in food intake, a change in accident rate, a change in respiratory rate, and a change in heart rate.

An information processing apparatus according to an embodiment of the present disclosure is an information processing apparatus including a controller, wherein the controller is configured to acquire an ingestion history of ingested substances and a change in health level of a resident in a facility, quantitatively analyze an effect on health level for each ingested substance or combination of ingested substances based on an ingestion history and change in health level of a plurality of residents, and output information on the effect on health level for each ingested substance or combination of ingested substances, and the change in health level includes at least one of a change in amount of sleep, a change in food intake, a change in accident rate, a change in respiratory rate, and a change in heart rate.

An information processing apparatus according to an embodiment of the present disclosure is an information processing apparatus including a controller, wherein the controller is configured to acquire an ingestion history of ingested substances and a change in health level of a resident in a facility, and output, as information on an effect on health level for each ingested substance or combination of ingested substances, a screen displaying each change in health level and an ingestion period of an ingested substance or combination of ingested substances for an individual resident, and the change in health level includes at least one of a change in amount of sleep, a change in food intake, a change in accident rate, a change in respiratory rate, and a change in heart rate.

A system according to an embodiment of the present disclosure is a system including the aforementioned information processing apparatus and a plurality of terminal apparatuses, wherein the information processing apparatus is configured to transmit, to the terminal apparatus, information on the effect on health level for each ingested substance or combination of ingested substances, and the terminal apparatus is configured to output the received information on the effect on health level.

A program according to an embodiment of the present disclosure is a program configured to cause a computer to execute operations, the operations including acquiring an ingestion history of ingested substances and a change in health level of a resident; quantitatively analyzing an effect on health level for each ingested substance or combination of ingested substances based on an ingestion history and change in health level of a plurality of residents; and outputting information on the effect on health level for each ingested substance or combination of ingested substances, wherein the change in health level includes at least one of a change in amount of sleep, a change in food intake, a change in accident rate, a change in respiratory rate, and a change in heart rate.

### Advantageous Effect

According to an embodiment of the present disclosure, technology related to maintaining the physical condition of residents in a residential facility is improved.

### BRIEF DESCRIPTION OF THE DRAWINGS

In the accompanying drawings:

FIG. 1 is a diagram illustrating a schematic configuration of a system according to an embodiment of the present disclosure;

FIG. 2 is a block diagram illustrating a schematic configuration of an information processing apparatus;

FIG. 3 is a block diagram illustrating a schematic configuration of a terminal apparatus;

FIG. 4 is a flowchart illustrating operations of the information processing apparatus according to an embodiment of the present disclosure;

FIG. 5 is a diagram illustrating an example of a user interface pertaining to outputting information on the effects of an ingested substance on health level;

FIG. 6 is a diagram illustrating an example of a user interface pertaining to a resident's health level;

FIG. 7 is a diagram illustrating an example of a user interface pertaining to a resident's health level;

FIG. 8 is a diagram illustrating an example of a user interface pertaining to a resident's health level;

FIG. 9 is a diagram illustrating an example of a user interface pertaining to a resident's health level;

FIG. 10 is a diagram illustrating an example of a user interface pertaining to a resident's health level;

FIG. 11 is a diagram illustrating an example of a user interface pertaining to a resident's health level;

FIG. 12 is a diagram illustrating an example of a user interface pertaining to a resident's health level;

FIG. 13 is a diagram illustrating an example of a user interface pertaining to a list of prescriptions; and

FIG. 14 is a diagram illustrating an example of a user interface pertaining to medication information.

### DETAILED DESCRIPTION

Embodiments of the present disclosure are described below with reference to the drawings.

### Overview of Embodiment

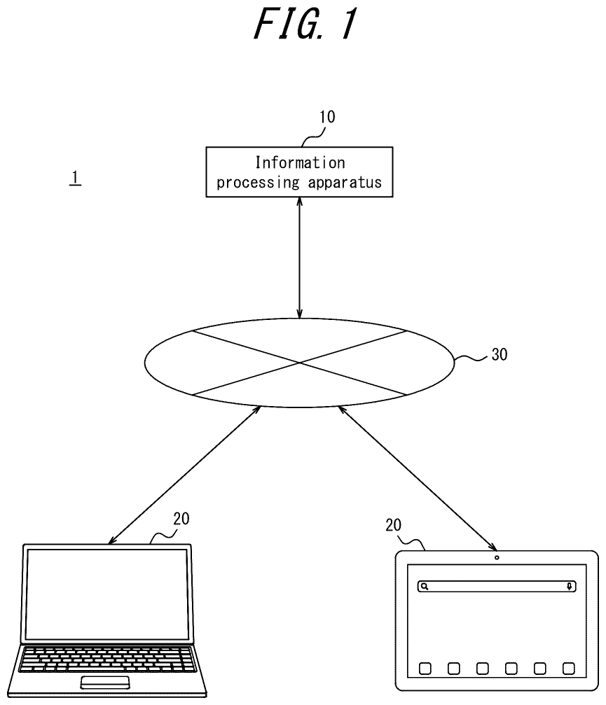

With reference to FIG. 1, an overview of a system 1 according to an embodiment of the present disclosure is described. The system 1 includes an information processing apparatus 10 and a plurality of terminal apparatuses 20. The information processing apparatus 10 and the plurality of terminal apparatuses 20 are, for example, communicably connected to a network 30 that includes the Internet, a mobile communication network, and the like.

The information processing apparatus 10 is, for example, a computer such as a server apparatus. For example, the information processing apparatus 10 is a server belonging to a cloud computing system or other computing system. The information processing apparatus 10 can communicate with the terminal apparatus 20 via the network 30. Although FIG. 1 illustrates an example of the system 1 including one information processing apparatus 10, this example is not limiting. The system 1 may include two or more information processing apparatuses 10.

The plurality of terminal apparatuses 20 is any electronic apparatus used by a user (such as a certified care worker, caregiver, or care manager, hereinafter also referred to simply as a “user”) of a nursing home, elderly care facility, or other residential facility (also referred to simply as a “facility”). For example, a general-purpose electronic device, such as a personal computer, smartphone, or tablet terminal, or a dedicated electronic device can be adopted as the terminal apparatus 20. Although FIG. 1 illustrates an example of the system 1 including two terminal apparatuses 20, the number of terminal apparatuses 20 may be freely determined.

First, an overview of the present embodiment is described, with details to be described later. The information processing apparatus 10 acquires, for example from the terminal apparatus 20, an ingestion history of ingested substances and a change in health level of a resident in a facility during a predetermined period. The information processing apparatus 10 then quantitatively analyzes the effect on the health level for each ingested substance or combination of ingested substances based on the inputted ingestion history and change in health level of a plurality of residents. The information processing apparatus 10 then outputs information on the effect on health level for each ingested substance or combination of ingested substances as the analysis result and transmits the information to each terminal apparatus 20, for example.

The terminal apparatus 20 can transmit, to the information processing apparatus 10, information on the ingestion history of ingested substances and the change in health level of the residents of a facility. The terminal apparatus 20 can also receive, from the information processing apparatus 10, the information on the effect on health level for each ingested substance or combination of ingested substances and can display the information.

In this way, according to the present embodiment, the system 1 (information processing apparatus 10) can output information on the effect on health level for each ingested substance or combination of ingested substances. The user can thereby know each ingested substance or combination of ingested substances that is likely to affect the health level, the degree of effect, and the like. Therefore, technology for maintaining the physical condition of residents in a residential facility is improved in that a resident's poor physical condition or an accident can be predicted and prevented.

Next, each of the components of the system 1 will be described in detail.

### Configuration of Information Processing Apparatus

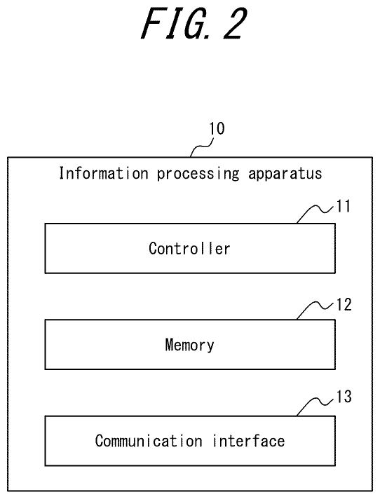

As illustrated in FIG. 2, the information processing apparatus 10 includes a controller 11, a memory 12, and a communication interface 13.

The controller 11 includes at least one processor, at least one dedicated circuit, or a combination thereof. The processor may, for example, be a general-purpose processor, such as a central processing unit (CPU) or graphics processing unit (GPU), or a dedicated processor specialized for particular processing. The dedicated circuit may, for example, be a field-programmable gate array (FPGA) or an application specific integrated circuit (ASIC). The controller 11 executes processing related to operation of the information processing apparatus 10 while controlling each component of the information processing apparatus 10.

The memory 12 includes at least one semiconductor memory, at least one magnetic memory, at least one optical memory, or a combination of at least two of these. The semiconductor memory is, for example, random access memory (RAM) or read only memory (ROM). The RAM is, for example, static random access memory (SRAM) or dynamic random access memory (DRAM). The ROM is, for example, electrically erasable programmable read only memory (EEPROM). The memory 12 functions as, for example, a main memory, an auxiliary memory, or a cache memory. The memory 12 stores programs and data to be used for operation of the information processing apparatus 10 and data resulting from operation of the information processing apparatus 10. The information stored in the memory 12 may, for example, be updated with information acquired from the network 30 via the communication interface 13.

The communication interface 13 includes at least one interface, which connects to the network 30, for communication with an external destination.

The interface for communication may be an interface for wired communication or wireless communication. In the case of wired communication, the interface for communication may be a local area network (LAN) interface or a universal serial bus (USB), for example. In the case of wireless communication, the interface for communication may be an interface conforming to a mobile communication standard, such as Long Term Evolution (LTE), 4^{th} Generation (4G), or 5^{th} Generation (5G), or an interface conforming to short-range wireless communication such as Bluetooth® (Bluetooth is a registered trademark in Japan, other countries, or both). The communication interface 13 receives data for use in operation of the information processing apparatus 10 and transmits data resulting from operation of the information processing apparatus 10 to an external destination (for example, the terminal apparatus 20).

Although the information processing apparatus 10 in FIG. 2 of the present embodiment is not depicted with an input interface and output interface, the information processing apparatus 10 may include non-illustrated input and output interfaces. In other words, in addition to receiving (input) and transmitting (output) information via the communication interface 13, information may be inputted and outputted by an input interface and output interface included in the information processing apparatus 10.

The functions of the information processing apparatus 10 are implemented by a processor corresponding to the controller 11 executing a program according to the present embodiment. In other words, the functions of the information processing apparatus 10 are implemented by software. The program causes a computer to function as the information processing apparatus 10 by causing the computer to execute the operations of the information processing apparatus 10. In other words, the computer functions as the information processing apparatus 10 by executing the operations of the information processing apparatus 10 in accordance with the program.

The program according to the present embodiment can be recorded on a computer readable recording medium. Computer readable recording media include non-transitory computer readable recording media, examples of which are a magnetic recording apparatus, an optical disc, a magneto-optical recording medium, and a semiconductor memory. The program is, for example, distributed by the sale, transfer, or lending of a portable recording medium such as a digital versatile disk (DVD) or a compact disk read only memory (CD-ROM) on which the program is recorded. The program may also be distributed by storing the program in the storage of an external server and transmitting the program from the external server to another computer. The program may also be provided as a program product.

A portion or all of the functions of the information processing apparatus 10 may be implemented by a dedicated circuit corresponding to the controller 11. In other words, a portion or all of the functions of the information processing apparatus 10 may be implemented by hardware.

### Configuration of Terminal Apparatus

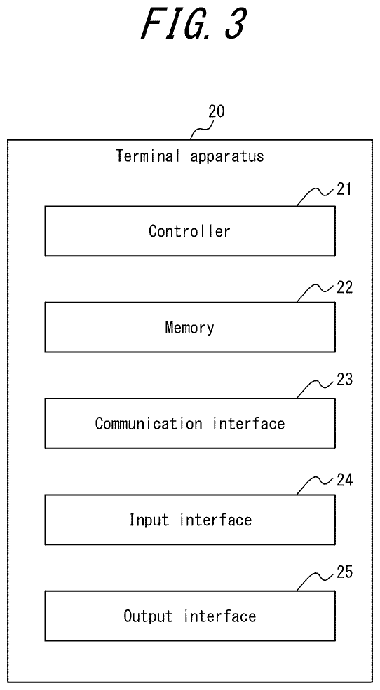

As illustrated in FIG. 3, the terminal apparatus 20 includes a controller 21, a memory 22, a communication interface 23, an input interface 24, and an output interface 25.

The controller 21 includes at least one processor, at least one dedicated circuit, or a combination thereof. The processor may be a general-purpose processor, such as a CPU or GPU, or a dedicated processor specialized for particular processing. The dedicated circuit is, for example, an FPGA or an ASIC. The controller 21 executes processing related to operations of the terminal apparatus 20 while controlling each component of the terminal apparatus 20.

The memory 22 includes at least one semiconductor memory, at least one magnetic memory, at least one optical memory, or a combination of at least two of these. The semiconductor memory is, for example, RAM or ROM. The RAM is, for example, SRAM or DRAM. The ROM is, for example, EEPROM. The memory 22 functions as, for example, a main memory, an auxiliary memory, or a cache memory. The memory 22 stores programs and data to be used for operation of the terminal apparatus 20 and data resulting from operation of the terminal apparatus 20. The information stored in the memory 22 may, for example, be updated with information acquired from the network 30 via the communication interface 23.

The communication interface 23 includes at least one interface, which connects to the network 30, for communication with an external destination. The interface for communication may be an interface for wired communication or wireless communication. In the case of wired communication, the interface for communication may be a LAN interface or a USB, for example. In the case of wireless communication, the interface for communication may be an interface conforming to a mobile communication standard, such as LTE, 4G, or 5G, or an interface conforming to short-range wireless communication such as Bluetooth®. The communication interface 23 receives, from the information processing apparatus 10, data for use in operation of the terminal apparatus 20 and transmits data resulting from operation of the terminal apparatus 20, for example.

The input interface 24 includes at least one interface for input. The interface for input is, for example, physical keys such as a keyboard, capacitive keys, a pointing device, or a touchscreen integrally provided with a display. The interface for input may, for example, be a microphone that receives audio input, a camera that receives gesture input, an optical character recognition (OCR) reader that works with a camera, or the like. The input interface 24 receives an operation for inputting data used in operation of the terminal apparatus 20. Instead of being provided in the terminal apparatus 20, the input interface 24 may be connected to the terminal apparatus 20 as an external input device. Any appropriate connection method can be used, such as USB, High-Definition Multimedia Interface (HDMI®) (HDMI is a registered trademark in Japan, other countries, or both), or Bluetooth®.

The output interface 25 includes at least one interface for output. The interface for output is, for example, a display that outputs information as images, a speaker that outputs information as audio, or the like. The display may, for example, be a liquid crystal display (LCD) or an organic electro luminescence (EL) display. The output interface 25 outputs and displays data received from the information processing apparatus 10, data resulting from operation of the terminal apparatus 20, or the like. Instead of being provided in the terminal apparatus 20, the output interface 25 may be connected to the terminal apparatus 20 as an external output device. Any appropriate connection method can be used, such as USB, HDMI®, or Bluetooth®.

The functions of the terminal apparatus 20 are implemented by a processor corresponding to the controller 21 executing a program according to the present embodiment. In other words, the functions of the terminal apparatus 20 are implemented by software. The program causes a computer to function as the terminal apparatus 20 by causing the computer to execute the operations of the terminal apparatus 20. In other words, the computer functions as the terminal apparatus 20 by executing the operations of the terminal apparatus 20 according to the program.

A portion or all of the functions of the terminal apparatus 20 may be implemented by a dedicated circuit corresponding to the controller 21. In other words, a portion or all of the functions of the terminal apparatus 20 may be implemented by hardware.

### Operations of Information Processing Apparatus

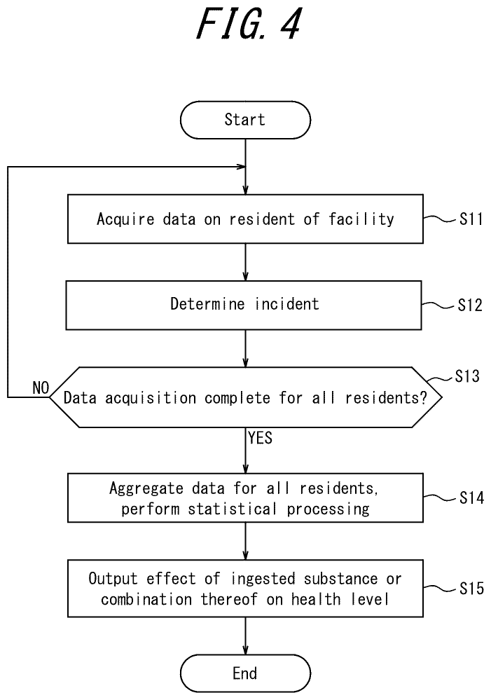

With reference to FIG. 4, operations of the information processing apparatus 10 according to the present embodiment are now described. FIG. 4 is a flowchart illustrating an example of a method executed by the information processing apparatus 10 according to the present embodiment.

Step S11: the controller 11 of the information processing apparatus 10 acquires, via the communication interface 13 or from other input means, data on an ingestion history of ingested substances and a change in health level of a resident. Here, the ingested substance includes at least one of a prescription drug (ethical drug), a medication requiring guidance, an over-the-counter medication, a quasi-drug, and a food product. Medications requiring guidance are medications that can only be purchased after receiving face-to-face guidance and information from a pharmacist. Over-the-counter medications are, for example, common cold medicines, fever-reducing analgesics, gastrointestinal analgesics, and the like. Quasi-drugs include, for example, drinks and supplements. Food products include ingredients ingested at daily meals and food items consumed between meals. The change in health level includes at least one of a change in amount of sleep, a change in food intake, a change in accident rate, a change in respiratory rate, and a change in heart rate of the resident.

A method of acquiring various types of data is now described. Information on prescription drugs (ethical drugs), including the drug and its dosage, can be acquired from each resident's prescription. Information on prescription drugs may be acquired not only from prescriptions written by physicians, but also from drug information forms, drug notebooks, or other similar documents (hereinafter referred to as “prescription or the like”). Data on the prescription or the like can be acquired by various means, such as keyboard input of the information on the prescription or the like from the input interface 24 of the terminal apparatus 20, image capture of a document containing drug information, reading of two-dimensional barcode information, or OCR reading of a drug notebook, and the data can be transmitted and inputted to the information processing apparatus 10. Prescription data may also be provided as electronic data by hospitals, pharmacies, and the like and directly inputted into the information processing apparatus 10. Data on medications or quasi-drugs other than ethical drugs can be inputted by the user from the terminal apparatus 20. Furthermore, the information processing apparatus 10 may acquire the medicinal effect, risk category, and the like of each medication from an external database via the communication interface 13.

Data on ingested food may be inputted from the terminal apparatus 20 for individually consumed food items, or information on the food intake at each meal (dietary intake) and information on the menu at each meal for each resident may be inputted to the information processing apparatus 10, and the food and nutritional components ingested and the intake may be determined from both sets of information. Data on beverages can also be inputted from the terminal apparatus 20 for the type and amount ingested by each resident individually. The information on the food intake of each resident at each meal is also used as basic data to acquire changes in the food intake. In other words, changes in the food intake can be acquired by recording the food intake of a resident at each meal and storing this information in the system 1 (information processing apparatus 10). For example, by recording and inputting information on the percentage of each main dish and side dish ingested by each resident at each meal, i.e., breakfast, lunch, and dinner, each day, the information processing apparatus 10 can ascertain the average food intake by each resident and the daily rate of change.

Changes in the amount of sleep of a resident can be acquired by measuring the resident's daily amount of sleep. The amount of sleep of the resident can, for example, be measured using a commercially available sleep monitor (an apparatus that measures sleep duration by detecting body movements and the like during sleep), and this data on sleep duration can be transmitted to the information processing apparatus 10.

Regarding changes in respiratory rate and changes in heart rate, the respiratory rate and heart rate are preferably acquired while the resident is at rest. There exist commercially available sleep monitors that can measure the respiratory rate and heart rate of a resident lying in bed at the same time as the amount of sleep, and this data on the respiratory rate and heart rate data can be transmitted to the information processing apparatus 10 and used as data indicating the health level. The resident's respiratory rate and heart rate are not limited to being measured by a sleep monitor and may, for example, be continuously measured by a device such as a wearable wristband. The measured data may be transmitted from the worn device to the information processing apparatus 10 in real time.

Changes in accident rates can be acquired by recording and storing the date, time, and content of accidents that occur to residents in a facility. The most common types of accidents include slips/falls, as well as unsteadiness, memory impairment, delirium, depression, urinary disorders, urinary incontinence, and the like. In particular, regarding slips/falls, fall information can be immediately transmitted to the information processing apparatus 10 or terminal apparatus 20 by having the resident carry a fall detection sensor. This enables monitoring of each resident's safety while accident data is accumulated.

In addition to changes in the resident's amount of sleep, changes in food intake, changes in accident rate, changes in respiratory rate, and changes in heart rate, data on the resident's body temperature and body weight may be used as data on changes in health level. For example, it is also useful to record data on the resident's body temperature and body weight daily and compare the data to changes in food intake.

Step S12: the controller 11 of the information processing apparatus 10 determines whether an incident (abnormality or event) has occurred based on the acquired data on the change in health level of the resident. Specifically, the controller 11 determines that an incident has occurred when the change in the health level satisfies (corresponds to) a predetermined condition. In a case in which an incident is determined to have occurred, the controller 11 preferably outputs an alert (warning) to draw attention.

The condition for determining an incident can be set as appropriate for each health level item. First, regarding a change in food intake, an incident is determined to have occurred when a sudden decrease in food intake (dietary intake) is observed, which triggers an alert. For example, when a certain resident's average food intake of main dishes and side dishes over the past (most recent) two days is less than 20% of the total provided meal, or when the rate of decrease is 30% or more compared to the resident's prior average food intake, the controller 11 determines that an incident has occurred. The numerical value or the like of the rate of decrease may be adjusted as necessary. Different conditions may also be set as incidents. The conditions may also include the resident's age, sex, level of nursing care, or the like (hereinafter referred to as “attributes”). In this way, the conditions for determining incidents related to the food intake can be adjusted according to a resident's attributes.

As for changes in sleep quantity, two incidents can be set: excessive sleep and insufficient sleep. For example, the controller 11 determines that an incident of a tendency to oversleep (excessive sleep) has occurred when the amount of sleep of a resident over the past (most recent) 2 days has increased by 35% or more compared to the average amount of sleep over the previous 30 days, and determines that an incident of a tendency towards insomnia (insufficient sleep) has occurred when the amount of sleep has decreased by 35% or more compared to the average amount of sleep over the previous 30 days. The numerical value or the like of the rate of increase or decrease may be adjusted as necessary. Different conditions may also be set as incidents. A resident's attributes may also be included in the conditions. In this way, the conditions for determining incidents related to the amount of sleep can be adjusted according to a resident's attributes.

As for changes in the rate of accidents, an incident may be determined to have occurred based on the rate of accidents that occurred during a certain period of time, but the occurrence of a slip/fall is preferably determined as a single incident. Alternatively, the occurrence of an accident in a broader sense (such as unsteadiness, memory impairment, delirium, depression, urinary disorders, and urinary incontinence, in addition to slips/falls) may be determined to be one incident.

As for changes in respiratory rate, the controller 11 immediately determines that an incident has occurred in a case in which an increase or decrease in respiratory rate exceeding a medically acceptable range of variation occurs relative to the average respiratory rate of a resident. Similarly, with regard to changes in heart rate, the controller 11 immediately determines that an incident has occurred in a case in which an increase or decrease in respiratory rate exceeding a medically acceptable range of variation occurs relative to the average respiratory rate of a resident. Since fluctuations in respiratory rate and heart rate are often associated with physical anomalies, real-time data acquisition and determination of incidents are preferred. Multiple levels of incident determination may also be used. For example, Level 1 (caution level) and Level 2 (danger level) determination conditions can be set. The range of variation in respiratory rate and heart rate that serves as a determination condition for an incident may be set appropriately in consideration of a resident's attributes.

Step S13: the controller 11 of the information processing apparatus 10 determines whether data acquisition for all residents has been completed. In a case in which data acquisition for all residents has not completed (step S13: NO), the process returns to step S11, and data is acquired for the next resident. In a case in which data acquisition for all residents has been completed (step S13: YES), the process proceeds to step S14. In the present embodiment, all residents refers to all of the residents for whom data input (data update) is necessary at that moment of processing, not necessarily all residents of the residential facility.

Step S14: in a case in which it is determined in step S13 that data acquisition for all residents has been completed (step S13: YES), the controller 11 aggregates the data for all residents and quantitatively analyzes the effect on the health level for each ingested substance or combination of ingested substances. This quantitative analysis may be performed by general statistical processing, or a neural network model may be created in the information processing apparatus 10, for example, to perform a trend analysis or the like of the ingested substances or combinations thereof using machine learning.

Step S15: the controller 11 of the information processing apparatus 10 outputs the effects of the ingested substance or combination of ingested substances on the health level based on the result of the statistical processing performed in step S14. For example, the controller 11 outputs the results by transmitting the results to the terminal apparatus 20 in response to an inquiry from the terminal apparatus 20 (search process or the like). Information on changes in the health level of each resident may also be outputted. Furthermore, in a case in which there is a resident for whom an incident has been determined, the controller 11 may notify the terminal apparatus 20 of the user in charge of that resident, or the terminal apparatus 20 that manages all of the residents, with information about the occurrence of the incident as an alert. The process then terminates.

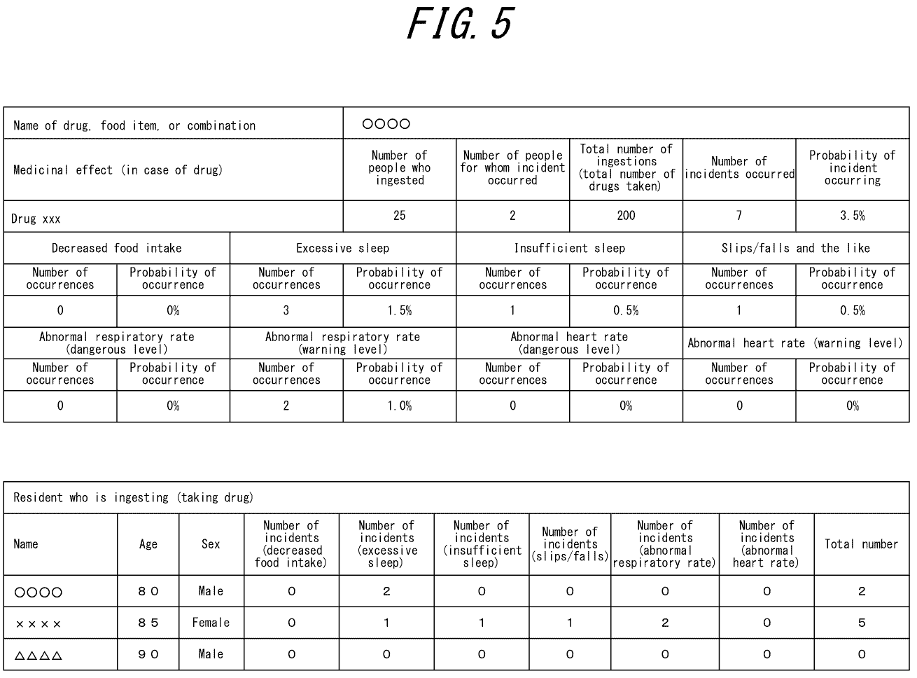

The information on the effect on health level for each ingested substance or combination of ingested substances as outputted by the information processing apparatus 10 will be described based on an example of a screen displayed on the output interface 25 of the terminal apparatus 20. FIG. 5 is a diagram illustrating an example of a user interface pertaining to outputting information on the effects of an ingested substance on the health level.

In the example in FIG. 5, relevant information regarding a certain drug, food item, or combination thereof is consolidated and displayed on a single screen. First, the name of the drug or food item (or a plurality of names in the case of a combination) is displayed as the “name of drug, food item, or combination”, and in the case of a drug, the medicinal effect is also displayed. The number of people who have ingested the drug or food item to date, the number of people for whom an incident occurred among those ingesting, the total number of ingestions (total number of drugs taken in the case of drugs), the number of incidents that occurred during the ingestion period, and the probability of an incident occurring (number of incidents/total number of ingestions) are displayed as information on the effects of the ingested substance on the health level. Furthermore, a breakdown of the content of the incidents is displayed, illustrating, for example, the number of incidents and probability of occurrence for each of the following items: decreased food intake, excessive sleep, insufficient sleep, slips/falls, respiratory rate, and heart rate. In addition, information on residents who have ingested (taken) the corresponding drug or food item may be displayed as reference information. For example, for each resident, the number of incidents may be displayed for each of the following items: decreased food intake, excessive sleep, insufficient sleep, slips/falls, respiratory rate, and heart rate. In this way, the information on the effect on health level for each ingested substance or combination of ingested substances includes statistical information on the incident. The statistical information on incidents illustrated in FIG. 5 is statistical information for all residents, but this configuration is not limiting. For example, the statistical information on incidents may include statistical information targeting residents with a predetermined attribute among all residents. Specifically, the statistical information may indicate, for example, the number of people for whom incidents occurred, the probability of occurrence, and the like for males in their 80s among all residents. As another example, the statistical information may indicate the number of people for whom incidents occurred, the probability of occurrence, and the like by sex. In this way, the correlation between a certain drug or the like and a resident with a predetermined attribute can be ascertained.

The output screen of information on the effects of an ingested substance on the health level in FIG. 5 can be set up so to be displayable by any means and by any screen transition. For example, the user can transition to the output screen of FIG. 5 by directly entering the name of the drug or food item, or a combination, on the input screen for the name of the ingested substance displayed on the terminal apparatus 20, or by selecting from a list of ingested substances displayed on the screen. Alternatively, the output screen illustrated in FIG. 5 can be transitioned to by extracting ingested substances that correspond to a conditional formula for an incident (number of incidents equal to or greater than X, probability of incident of decreased food intake equal to or greater than Y%, or the like) and then selecting from a list of the extracted ingested substances. This screen transition is merely an example. Information on the effects of ingested substances on the health level may be displayed in other ways.

Furthermore, by selecting a resident (such as by clicking on the screen) on the screen in FIG. 5 from a list of residents who are ingesting (taking) a drug or food item, information on changes in the health level of the corresponding individual resident may be displayed. Next, an example of an output screen of information on changes in an individual resident's health level will be described.

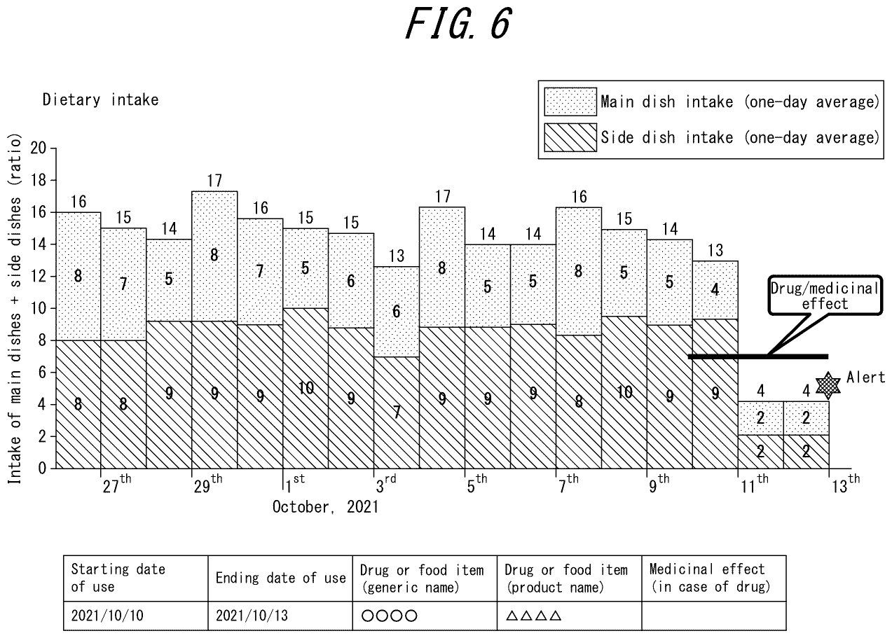

FIG. 6 is an example of a user interface pertaining to a resident's (individual's) health level. FIG. 6 illustrates the change in an individual resident's dietary intake (food intake). The horizontal axis of the graph indicates the year, month, and date, and the vertical axis indicates the dietary intake for each day. Here, the meals are divided into main dishes and side dishes, and the ingested percentage of the total (serving size) for each is indicated by a number from 0 to 10. Therefore, the vertical axis is indicated by a number with a maximum value of 20. The data on the dietary intake is acquired for each meal, i.e., breakfast, lunch, and dinner, and the displayed graph illustrates the daily average value as a bar graph. In a case in which the dietary intake corresponds to an incident condition, an alert mark is preferably displayed on the graph for the corresponding day. FIG. 6 illustrates an example in which an incident was determined to have occurred, and an alert was displayed, based on a condition that the average food intake for main dishes and side dishes for the past (most recent) two days is 20% or less of the total meals served.

In FIG. 6, a line indicating an ingested substance (such as a medication being taken) that the corresponding resident is taking is displayed in overlap on the graph of the dietary intake. The line extends horizontally with a length corresponding to the ingestion period of the ingested substance. The name of the ingested substance (for example, the drug name or product name) may be displayed near the line, or the user interface may be such that the name of the ingested substance is displayed when the line is touched or the cursor is moved over the line. Furthermore, in the case of a drug, the display may be toggled between the name and the medicinal effect. In addition, a list of drugs and the like that the resident is taking may be displayed on the same screen as the graph. From this screen, the user can ascertain the effect of a certain ingested substance on the resident's dietary intake. The ingested substance is not limited to medications, and the display may also focus on quasi-drugs (drinks, supplements, and the like) and food items (for example, irritating ingredients).

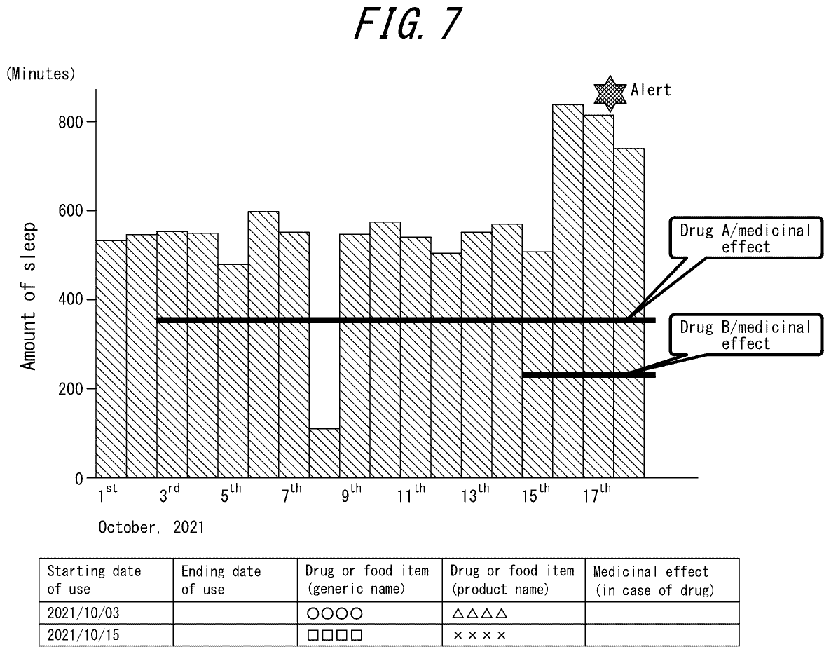

FIG. 7 is another example of a user interface pertaining to a resident's (individual's) health level. FIG. 7 illustrates the change in the amount of sleep for an individual resident. The horizontal axis of the graph indicates the year, month, and date, and the vertical axis indicates the amount of sleep for each day. Here, the daily amount of sleep measured by a sleep monitor is indicted in minutes. In a case in which the amount of sleep corresponds to an incident condition, an alert mark is preferably displayed on the graph for the corresponding day. FIG. 7 illustrates an example in which an incident of excessive sleep was determined to have occurred, and an alert was displayed, based on a condition that the amount of sleep over the past (most recent) two days increased by 35% or more compared to the average amount of sleep over the previous 30 days.

In FIG. 7, lines indicating ingested substances (such as a medication being taken) that the corresponding resident is taking are displayed in overlap on the graph of the amount of sleep. The lines extend horizontally with a length corresponding to the ingestion period of the ingested substance. The different line heights in FIG. 7 indicate that two different drugs are being taken. By indicating each drug with a different line from the start of use to the end of use (including continued use), the user can immediately ascertain how many types of medications the resident is taking and the danger of multiple drug administration. The name of the ingested substance (for example, the drug name or medicinal effect) may be displayed near each line, or the user interface may be such that the name of the ingested substance is displayed when the line is touched or the cursor is moved over the line. In addition, a list of drugs and the like that the resident is taking may be displayed on the same screen as the graph.

From the changes in amount of sleep in FIG. 7, it is possible to ascertain the resident's amount of sleep was affected after ingestion of a certain ingested substance (drug B), but in order to determine whether this is an effect of drug B alone or an interaction between drug A and drug B, it is necessary also to analyze the trends in the amount of sleep of other residents who are taking drug B. The information processing apparatus 10 uses statistical processing or machine learning to analyze the effect of such ingested substances or combination thereof on the health level.

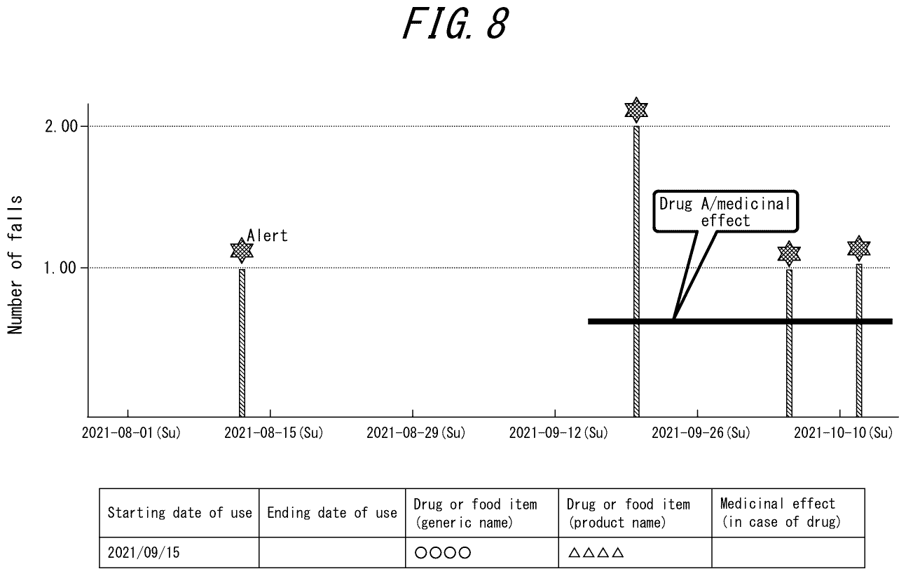

FIG. 8 is another example of a user interface pertaining to a resident's (individual's) health level. FIG. 8 illustrates the occurrence of accidents (falls) for an individual resident. The horizontal axis of the graph indicates the year, month, and date, and the vertical axis indicates the number of accidents. For accidents (falls), an alert mark is preferably displayed on the graph for the day on which the incident occurred, assuming that the occurrence of the accident itself constitutes an incident.

In FIG. 8, a line indicating an ingested substance (such as a medication being taken) that the corresponding resident is taking is displayed in overlap on the graph of accident occurrence. The line extends horizontally with a length corresponding to the ingestion period of the ingested substance. The name of the ingested substance (for example, the drug name or medicinal effect) may be displayed near the line, or the user interface may be such that the name of the ingested substance is displayed when the line is touched or the cursor is moved over the line. In addition, a list of drugs and the like that the resident is taking may be displayed on the same screen as the graph. From this screen, the user can ascertain the effect of a certain ingested substance on fall accidents for the resident.

In addition, data such as the resident's respiratory rate, heart rate, and body weight may also be displayed as a graph of daily changes, as in FIG. 7, along with a line extending horizontally to indicate the ingested substance (such as a medication being taken) that the corresponding resident is taking, so that the relationship with the ingested substance can be ascertained. Vital data for an individual resident (such as food intake, amount of sleep, respiratory rate, heart rate, body weight, and body temperature) may be aggregated for further display, in addition to FIGS. 6 to 8, of a screen of information to manage the resident's health status.

Although it has been described as being possible to transition from the output screen of information on the effects of the ingested substance on the health level in FIG. 5 to the output screen of information on changes in the health level of an individual resident (for example, FIGS. 6 to 8), an individual resident may be directly specified for the information processing apparatus 10 to output information on changes in the health level of that resident. For example, the user inputs an individual resident's name from the terminal apparatus 20, and the information processing apparatus 10 outputs personal information on that resident in response. Each of the screens in FIGS. 6 to 8 may be displayed by switching from each resident's personal information screen using a tab or the like.

The information, outputted by the information processing apparatus 10, on the effect on the health level for each ingested substance or combination of ingested substances may be outputted using a different user interface than the user interface in FIG. 5. For example, by aggregating the data of residents who are ingesting a certain ingested substance or combination of ingested substances, each change over time in health level after the start of ingestion of a certain ingested substance or combination of ingested substances may be displayed as an average value for the ingesting residents. In other words, for each ingested substance or combination thereof, a graph similar to each change in health level in FIGS. 6 to 8 may be outputted as an average value for the ingesting residents.

When an incident is determined, the information processing apparatus 10 may notify the terminal apparatus 20 of the user in charge of the resident for whom the incident has been determined, or the terminal apparatus 20 that manages all of the residents, with information about the occurrence of the incident as a push notification. The information processing apparatus 10 may directly transmit the screen (FIGS. 6 to 8) of information on changes in the health level of the corresponding resident, with the display of an alert added on, to the terminal apparatus 20 for output.

According to such a configuration, the system 1 (information processing apparatus 10) can output information on the effect on the health level for each ingested substance or combination thereof from the information on the overall ingested substances and changes in health level for the residents of the facility. The user can, for example, thereby know each ingested substance or combination of ingested substances that is likely to affect the health level. Therefore, technology for maintaining the physical condition of residents in a residential facility is improved in that a resident's poor physical condition or an accident can be predicted and prevented.

In the present embodiment, it is assumed that data on prescriptions and the like can be acquired by reading two-dimensional barcode information using the terminal apparatus 20, but two-dimensional barcode information can be read using various methods. Specifically, for example, data on prescriptions and the like may be acquired by reading a two-dimensional code, printed on the prescription or the like, for electronic drug notebooks. In this case, information not included in the two-dimensional code for electronic drug notebooks may be added to the input by the user. For example, resident facility information may be added by selection or other input by the user. In this case, the resident facility information may be transmitted from the terminal apparatus 20 to the information processing apparatus 10 together with the two-dimensional code information.

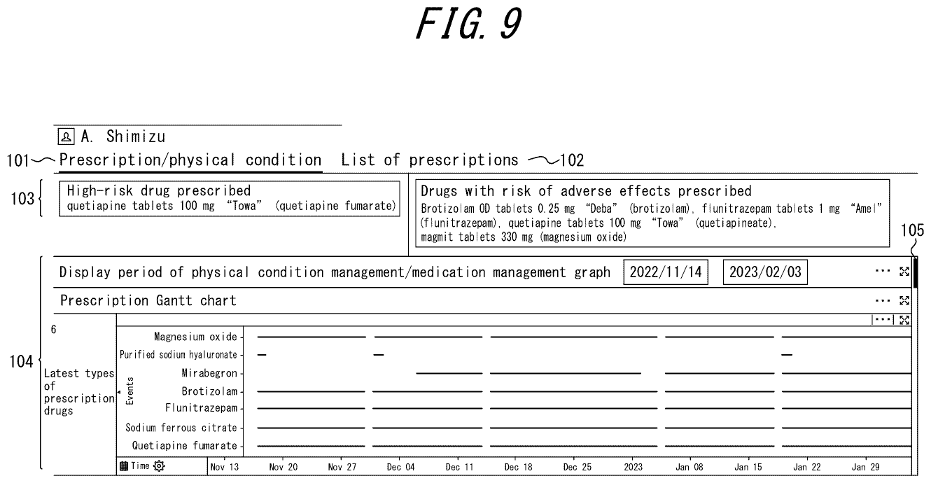

The user interface pertaining to a resident's (individual's) health level is not limited to the example in FIG. 6 or the like. FIGS. 9 to 12 illustrate other examples of a user interface pertaining to a resident's (individual's) health level. The user interface illustrated in FIG. 9 illustrates information pertaining to the health level of an individual resident (in this case, A. Shimizu). The user interface in FIG. 9 includes a selection tab 101, a selection tab 102, a notes column 103, an information display column 104, and a scroll bar 105. When the selection tab 101 is selected by the user, information indicating prescriptions and physical condition is displayed. When the selection tab 102 is selected by the user, information pertaining to the resident's prescriptions in general is displayed. FIG. 9 is the user interface that is displayed when the selection tab 101 is selected. The notes column 103 indicates notes about the resident's prescriptions. For example, in a case in which a medication with a risk exceeding a predetermined standard is prescribed to a resident, the information “High-risk drug prescribed” is indicated. As illustrated in FIG. 9, the notes column 103 may also list specific medication names. In a case in which a resident is prescribed a medication that exceeds a predetermined standard regarding the risk of adverse effects, the notes column 103 may also indicate the information “Drug with risk of adverse effects prescribed”. As illustrated in FIG. 9, the notes column 103 may also list specific medication names pertaining to drugs with a risk of adverse effects.

Various information is displayed in the information display column 104. The various information includes a prescription Gantt chart, and changes over time in dietary intake, breakfast intake, body weight, body temperature, fluid intake, stool output, sleep duration, and the like. The content illustrated in the information display column 104 can be changed by using the scroll bar 105 to change the display range of the information display column 104. In FIG. 9, a prescription Gantt chart is illustrated in the information display column 104. The prescription Gantt chart is a graph that illustrates the date and time of prescription of each medication via a Gantt chart. The horizontal axis of the Gantt chart indicates the date and time, and the vertical axis indicates each medication. The prescription Gantt chart enables the user easily to understand when each medication is prescribed.

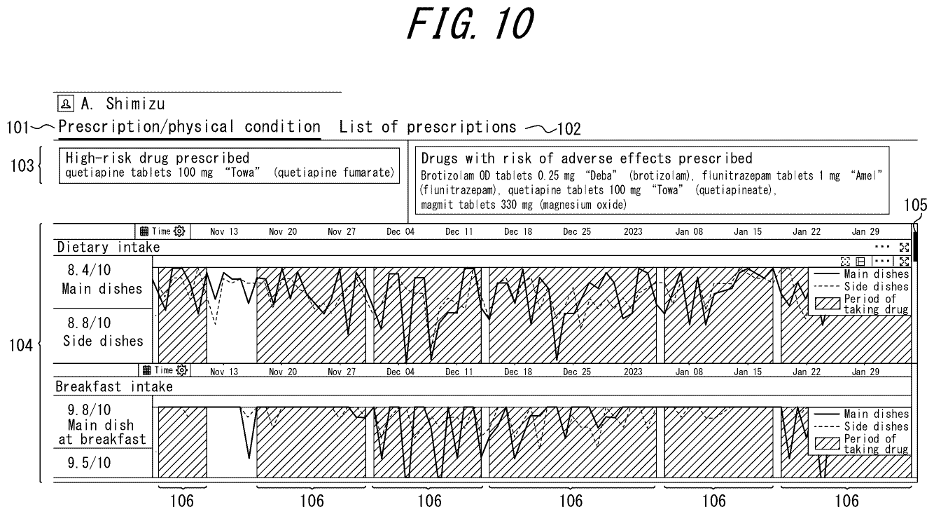

When the scroll bar 105 is operated as described above, the display range of the information display column 104 changes. FIG. 10 is an example of the display after the display range of the information display column 104 is changed by operation of the scroll bar 105. In FIG. 10, the information display column 104 illustrates the change over time in dietary intake and breakfast intake. The horizontal axis of the graph indicates the year, month, and date, and the vertical axis indicates the dietary intake and breakfast intake for each day. In addition, in FIG. 10, the ingestion period of medications that the resident is taking is indicated by areas 106, which illustrate the range corresponding to the ingestion period, overlaid on the graphs of the change over time in dietary intake and breakfast intake.

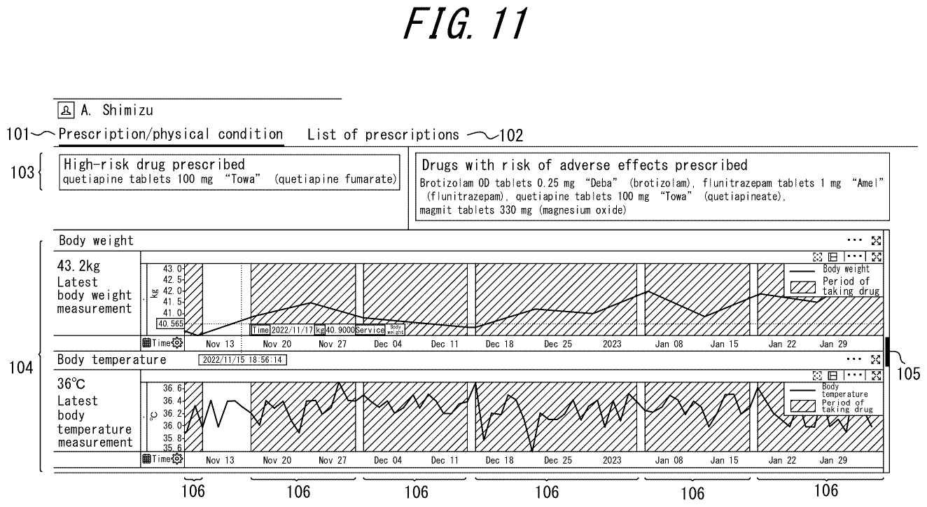

FIG. 11 is an example of the display after the display range of the information display column 104 is changed by further operation of the scroll bar 105. In FIG. 11, the change over time in body weight and body temperature is illustrated in the information display column 104. The horizontal axis of the graph indicates the year, month, and date, and the vertical axis indicates the body weight and body temperature on each day. In FIG. 11, the ingestion period of medications that the resident is taking is indicated by areas 106, which illustrate the range corresponding to the ingestion period, overlaid on the graphs of the change over time in body weight and body temperature, as in FIG. 10. This enables the user to ascertain the relationship between the health level and the ingested substances, such as medications, easily.

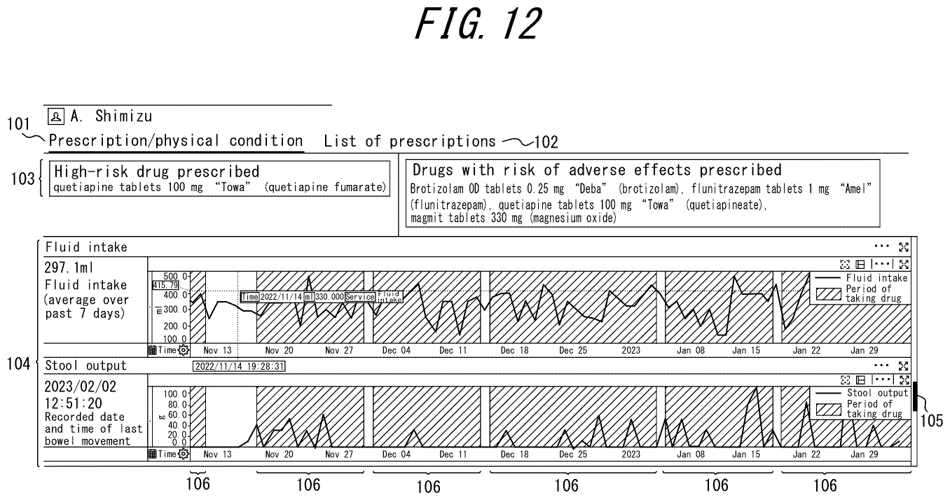

FIG. 12 is an example of the display after the display range of the information display column 104 is changed by further operation of the scroll bar 105. In FIG. 12, the change over time in fluid intake and stool output is illustrated in the information display column 104. The horizontal axis of the graph indicates the year, month, and date, and the vertical axis indicates the fluid intake and stool output for each day. In FIG. 12, the ingestion period of medications that the resident is taking is indicated by areas 106, which illustrate the range corresponding to the ingestion period, overlaid on the graphs of the change over time in fluid intake and stool output, as in FIG. 10. This enables the user to ascertain the relationship between the health level and the ingested substances, such as medications, easily.

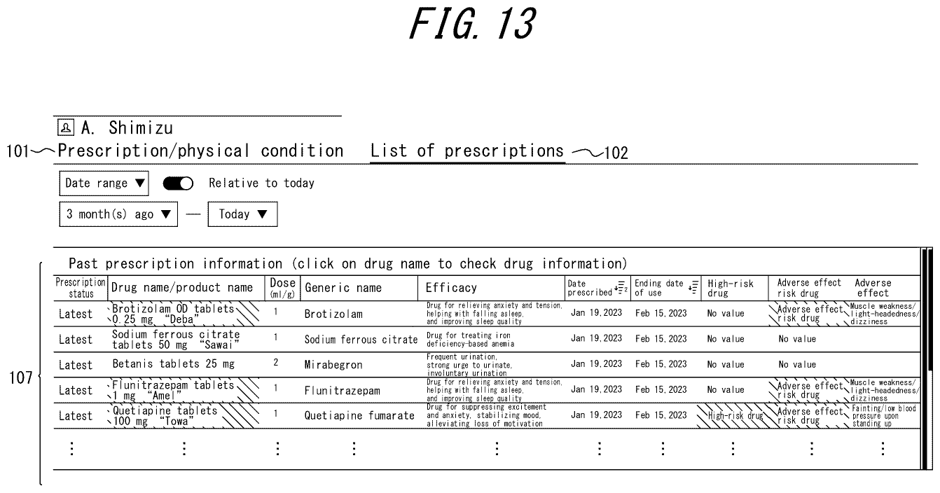

When the selection tab 102 in FIG. 9 is selected, information pertaining to the resident's prescriptions in general is displayed. FIG. 13 is an example of the user interface that is displayed when the selection tab 102 is selected. The user interface in FIG. 13 includes a selection tab 101, a selection tab 102, and list information 107. The selection tab 101 and the selection tab 102 are identical to those illustrated in FIG. 9. The list information 107 includes various information on the medications prescribed to the resident. The various information on the medications includes prescription status, drug name, dosage, generic name, efficacy, prescription date, starting date of use, high-risk drug flag, adverse effect risk drug flag, adverse effects, and the like. The high-risk drug flag is a flag indicating whether the medication entails a risk exceeding a predetermined standard. In the list information 107, a medication with a risk exceeding a predetermined standard is indicated as being a “high-risk drug”. If a medication with a high-risk drug flag is included, the fact that a high-risk drug is being prescribed is indicated in the above-described notes column 103. The adverse effect risk drug flag is a flag indicating whether a medication entails an adverse effect risk exceeding a predetermined standard. In the list information 107, a medication with an adverse effect risk exceeding a predetermined standard is indicated as being an “adverse effect risk drug”. If a medication with an adverse effect risk drug flag is included, the fact that an adverse effect risk drug is being prescribed is indicated in the above-described notes column 103.

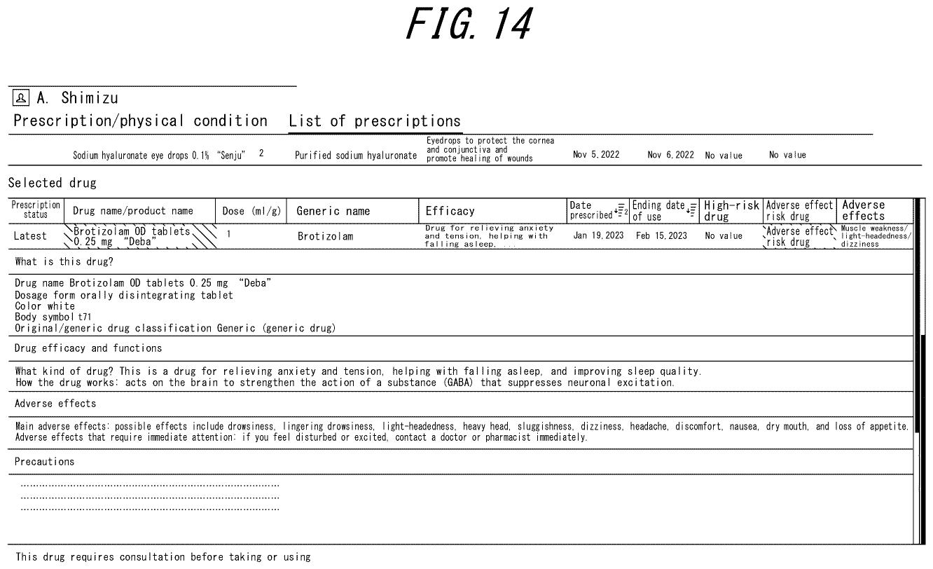

Each medication name in FIG. 13 may be selectable by the user by clicking or other means. In this case, when a certain medication name is selected, detailed information about the selected drug may be displayed. FIG. 14 is an example of a user interface when one of the medication names in the list in FIG. 13 is selected. As illustrated in FIG. 14, this user interface displays the content of the drug, efficacy and functions, adverse effects, precautions, and the need to consult before taking or using the drug. Such a user interface enables the user to ascertain detailed information about the drug easily.

Although the present disclosure is based on embodiments and drawings, it is to be noted that various changes and modifications may be made by those skilled in the art based on the present disclosure. Therefore, such changes and modifications are to be understood as included within the scope of the present disclosure. For example, the functions and the like included in the components, steps, and the like may be reordered in any logically consistent way. Furthermore, components, steps, and the like may be combined into one or divided.

For example, an embodiment in which the configuration and operations of the information processing apparatus 10 in the above-described embodiment are distributed to a plurality of computers that can communicate with each other is also possible. An embodiment in which some of the functions and components of the information processing apparatus 10 are provided in the terminal apparatus 20, for example, is also possible.

An embodiment in which a general-purpose computer, for example, is caused to function as the information processing apparatus 10 according to the above embodiment is also possible. Specifically, a program containing processing content for implementing the functions of the information processing apparatus 10 according to the above embodiment may be stored in the memory of a general-purpose computer, and the processor may read and execute the program. Accordingly, the present disclosure can also be embodied as a program executable by a processor or as a non-transitory computer readable medium storing the program.

### REFERENCE SIGNS LIST

1 System
10 Information processing apparatus
11 Controller
12 Memory
13 Communication interface
20 Terminal apparatus
21 Controller
22 Memory
23 Communication interface
24 Input interface
25 Output interface
30 Network
101, 102 Selection tab
103 Notes column
104 Information display column
105 Scroll bar
106 Area
107 List information

1. A method to be executed by an information processing apparatus, the method comprising:
acquiring an ingestion history of ingested substances and a change in health level of a resident in a facility;
quantitatively analyzing an effect on health level for each ingested substance or combination of ingested substances based on an ingestion history and change in health level of each of a plurality of residents; and
outputting information on the effect on health level for each ingested substance or combination of ingested substances based on a result of the analyzing, wherein
the change in health level includes at least one of a change in amount of sleep, a change in food intake, a change in accident rate, and a change in respiratory rate.

2. The method according to claim 1, further comprising:
determining that an incident has occurred when the change in health level of an individual resident satisfies a predetermined condition, wherein
the information on the effect on health level for each ingested substance or combination of ingested substances includes statistical information on the incident.

3. The method according to claim 1, further comprising outputting a screen displaying, for an individual resident, the change in health level and an ingestion period of an ingested substance or combination of ingested substances.

4. The method according to claim 1, wherein the ingested substance includes at least one of a prescription drug, a medication requiring guidance, an over-the-counter medication, a quasi-drug, and a food product.

5. The method according to claim 2, further comprising adjusting the condition for determining the incident in accordance with an attribute of the resident.

6. (canceled)

7. An information processing apparatus comprising a controller, wherein
the controller is configured to
acquire an ingestion history of ingested substances and a change in health level of a resident in a facility,
quantitatively analyze an effect on health level for each ingested substance or combination of ingested substances based on an ingestion history and change in health level of each of a plurality of residents, and
output information on the effect on health level for each ingested substance or combination of ingested substances based on a result of the analyzing, and
the change in health level includes at least one of a change in amount of sleep, a change in food intake, a change in accident rate, and a change in respiratory rate.

8. The information processing apparatus according to claim 7, wherein
the controller is configured to determine that an incident has occurred when the change in health level of an individual resident satisfies a predetermined condition, and
the information on the effect on health level for each ingested substance or combination of ingested substances includes statistical information on the incident.

9. The information processing apparatus according to claim 8, wherein
when determining that an incident has occurred regarding the change in health level of an individual resident,
the controller is configured to output information on an alert regarding the change in health level of the resident.

10. (canceled)

11. A system comprising the information processing apparatus according to claim 7 and a plurality of terminal apparatuses, wherein
the information processing apparatus is configured to transmit, to the terminal apparatus, information on the effect on health level for each ingested substance or combination of ingested substances, and
the terminal apparatus is configured to output the received information on the effect on health level.

12. A non-transitory computer readable medium storing a program configured to cause a computer to execute operations, the operations comprising:
acquiring an ingestion history of ingested substances and a change in health level of a resident in a facility;
quantitatively analyzing an effect on health level for each ingested substance or combination of ingested substances based on an ingestion history and change in health level of each of a plurality of residents; and
outputting information on the effect on health level for each ingested substance or combination of ingested substances based on a result of the analyzing, wherein
the change in health level includes at least one of a change in amount of sleep, a change in food intake, a change in accident rate, and a change in respiratory rate.

13. (canceled)

14. The method according to claim 2, wherein the statistical information includes number of people who ingested, number of people for whom incident occurred, total number of ingestions, number of incidents occurred, and probability of incident occurring.

15. The method according to claim 14, wherein the statistical information includes a breakdown of the incidents.

16. The method according to claim 1, further comprising:
outputting list information of residents ingesting each ingested substance or combination of ingested substances.
